(19) United States
(12) Patent Application Publication
(10) Pub. No.: US 2023/0309989 A1
(43) Pub. Date: Oct. 5, 2023

(54) Assembly and Method for Closing a Tissue Opening

(71) Applicant: Ethicon, Inc., Raritan, NJ (US)

(72) Inventors: Nicolo Garbin, Houston, TX (US); Steven Nguyen, Houston, TX (US); Abdul Umaru, Houston, TX (US); William Cohn, Houston, TX (US); Marcus Pantoja, Houston, TX (US)

(21) Appl. No.: 18/187,079

(22) Filed: Mar. 21, 2023

Related U.S. Application Data
(60) Provisional application No. 63/325,648, filed on Mar. 31, 2022.

(51) Int. Cl. A61B 17/04

(52) U.S. Cl. CPC A61B 17/0469; A61B 2017/06176

(57) ABSTRACT

An assembly includes an insertion device and a suture delivery device. The insertion device includes a longitudinal guide member extending from a proximal end to a distal tip. The tip includes an abutting structure movable between insertion and open positions. The structure is sized and shaped to contact tissue surrounding the tissue opening into which the insertion device has been inserted to maintain a desired position of the insertion device within the opening. The delivery device includes a body and a tube. The tube extends along a curved path configured so that, as the tube is rotated about the member, the tube penetrates tissue surrounding the opening into which the member has been inserted. A distal extension of the tube is configured so that, when the tube has been advanced distally to a distal-most position on the member, a distal end of the tube is proximal of the structure.

### PRIORITY CLAIM

The present application present disclosure claims priority to U.S. Provisional Patent Application Ser. No. 63/325,648 filed Mar. 31, 2022; the disclosure of which is incorporated herewith by reference.

### BACKGROUND

A port access incision may be made in a patient, e.g., an abdomen, for medical procedures including, e.g., laparoscopic, thoracoscopic or robotic surgeries. For example, an incision may be made in the abdomen so that a port, or trocar, may be implanted in the abdomen through which an operating physician can insert and withdraw instruments for performing the medical procedure. After the procedure in the abdomen has been completed, the port is generally removed and the incision is closed.

### SUMMARY

The present disclosure relates to an assembly for closing a tissue opening. The assembly includes an insertion device including a longitudinal guide member extending from a proximal end to a distal tip. The distal tip includes an abutting structure movable between an insertion position in which the abutting structure is flush with the distal tip and an open position in which the abutting structure is extended laterally from the distal tip. The abutting structure is sized and shaped to contact tissue surrounding the tissue opening into which the insertion device has been inserted to maintain a desired position of the insertion device within the tissue opening.

The assembly also includes a suture delivery device which has a body and a tube. The tube includes a suture channel extending therethrough. The tube is translatable and rotatable relative to the longitudinal guide member. The tube extends along a curved path configured so that, as the tube is rotated about the longitudinal guide member, the tube penetrates tissue surrounding the tissue opening into which the longitudinal guide member has been inserted. A distal extension of the tube is configured so that, when the tube has been advanced distally to a distal-most position on the longitudinal guide member, a distal end of the tube is proximal of the abutting structure.

In an embodiment, the assembly further includes a suture slidably received within the tube, the suture including a tissue anchoring distal tip extending distally out of the distal end of the tube, so that, as the tube is introduced into tissue, the tissue anchoring distal tip is driven into the tissue and retained in the tissue to anchor the suture in the tissue as the tube is withdrawn therefrom so that tightening the suture closes the tissue opening.

In an embodiment, the insertion device further comprises an actuator coupled to the abutting structure so that movement of the actuator in a first direction relative to the longitudinal guide member extends the abutting structure laterally into the open position and movement of the actuator in a second direction relative to the longitudinal guide member withdraws the abutting structure laterally into a closed position.

In an embodiment, the tube is configured so that, as the tube is advanced distally to the distal-most position relative to the longitudinal guide member, the tube is advanced through the tissue around the tissue opening around a full circumference of the tissue opening so that pulling the suture proximally while the suture is anchored in the tissue, produces a multi-layer helical closure of the tissue opening.

In an embodiment, the body includes a channel extending therethrough sized and shaped to slidably and rotatably receive the longitudinal guide member therein.

In an embodiment, the tube extends distally from the suture delivery device for a distance selected to ensure that, when the tube is inserted into tissue until an aspect of the suture delivery device contacts an outer surface of the tissue, the distal end of the tube will have penetrated the tissue to a desired depth that is proximal of the abutting structure.

In an embodiment, the suture includes a tissue anchoring barb at a distal end thereof.

In an embodiment, the actuator is coupled to an inner member slidably received within the longitudinal guide member, a distal end of the inner member being coupled to the abutting structure so that movement of the inner member relative to the longitudinal guide member moves the abutting structure between the open and closed positions.

In an embodiment, the abutting structure includes a plurality of projections rotatably coupled to the distal tip for rotating between the open and closed positions.

In an embodiment, the tube is helical and extends at least twice around a circumference of the body.

In an embodiment, the body of the suture delivery device is slidably coupled to the insertion device, the suture delivery device further comprising a handle slidably and rotatably coupled to the body, the tube being fixed to the handle.

In an embodiment, the body further comprises a helical guide recess for receiving the tube prior to and during implantation of the tube into the tissue.

In an embodiment, the suture delivery device further comprises a port into which liquid medication can be administered, the port connecting to the tube so that the liquid medication travels through the tube and is released into the tissue via holes in the tube.

In an embodiment, the suture delivery device further comprises a clamp for locking the suture delivery device at a location along the insertion device via a locking knob.

In an embodiment, the suture delivery device comprises a mechanism for detaching a tissue anchoring distal tip carrying a suture from the tube in which the tissue anchoring distal tip is carried.

In addition, the present disclosure relates to a method for closing a tissue opening. The method includes inserting into the tissue opening an insertion device including a longitudinal guide member extending from a proximal end to a tip at a distal end thereof with an abutting structure of the tip in an insertion position in which the abutting structure is flush with the distal tip until the tip has passed fully through a thickness of the tissue surrounding the tissue opening; after the tip has passed fully through the thickness of the tissue surrounding the tissue opening, moving the abutting structure to an open position in which the abutting structure is extended laterally away from the tip; withdrawing the longitudinal guide member proximally until the abutting structure contacts an inner surface of the tissue surrounding the tissue opening; rotating and moving distally along the longitudinal guide member a tube extending distally from a suture delivery device along a curved path so that, as the tube is rotated relative to the longitudinal guide member, the tube penetrates tissue surrounding the tissue opening, a distal extension of the tube being configured so that, when the tube has been advanced distally to a distal-most position on the longitudinal guide member, a distal end of the tube is proximal of the abutting structure, the tube including a suture slidably received therein and including a tissue anchor at a distal end thereof; and withdrawing the tube from the tissue while the tissue anchor remains lodged in the tissue so that, as the tube is withdrawn from the tissue, the suture slides out of the tube to remain within the tissue around the tissue opening.

In an embodiment, the method further includes pulling the suture proximally to close the tissue opening.

In an embodiment, the method further includes after the tip has passed fully through the thickness of the tissue surrounding the tissue opening, moving an actuator coupled to the abutting structure in a first direction relative to the longitudinal guide member to extend the abutting structure laterally into the open position.

In an embodiment, the method further includes after rotating and moving a body of the suture delivery device distally along the longitudinal guide member to advance the tube into the tissue, moving the actuator in a second direction relative to the longitudinal guide member to withdraw the abutting structure laterally into a closed position.

In an embodiment, as the tube is advanced distally to the distal-most position relative to the longitudinal guide member, the tube is advanced through the tissue around the tissue opening around a full circumference of the tissue opening.

### BRIEF DESCRIPTION OF THE DRAWINGS

FIG. 1 shows a first exemplary assembly including an insertion device and a suture delivery device.

FIG. 2 shows the insertion device of FIG. 1.

FIG. 3 shows a distal tip of the insertion device of FIG. 1 with projections in a closed position.

FIG. 4 shows the distal tip of the insertion device of FIG. 1 with the projections in an open position.

FIG. 5 shows the suture delivery device of FIG. 1.

FIG. 6a shows a sharp distal anchor tip of a barbed suture extending from a distal end of a helical tube of the suture delivery device of FIG. 1.

FIGS. 6b-c show an alternative embodiment of the suture delivery device of FIG. 1 comprising a slotted helical wire instead of a helical tube.

FIG. 7 shows the distal tip of the insertion device of FIGS. 1-6 inserted into an incision in a tissue wall with the projections retracted.

FIGS. 8-9 show the distal tip of the insertion device of FIGS. 1-6 inserted into the incision in the tissue wall with the projections extended.

FIG. 10 shows the projections of the distal tip of the insertion device of FIGS. 1-6 brought into contact with an inner surface of the tissue wall.

FIG. 11 shows the suture delivery device of FIGS. 1-6 slid into position adjacent to an outer surface of the tissue wall.

FIG. 12 shows the suture delivery device of FIGS. 1-6 advanced through the tissue wall.

FIG. 13 shows the suture delivery device of FIGS. 1-6 advanced through the tissue wall with the insertion device withdrawn.

FIGS. 14-16 show the barbed suture of the suture delivery device of FIGS. 1-6 advanced through the tissue wall with a body and the helical tube of the suture delivery device withdrawn.

FIG. 17 shows the barbed suture of the suture delivery device of FIGS. 1-6 pulled proximally to close the incision.

FIGS. 18-19 show the suture of the suture delivery device of FIGS. 1-6 in the position.

FIG. 20 shows an exemplary method for closing a port access incision in the tissue wall using the assembly described in FIGS. 1-6.

FIG. 21 shows a second exemplary assembly including an insertion device and a suture delivery device.

FIG. 22 shows the insertion device of FIG. 21.

FIG. 23 shows the suture delivery device of FIG. 21.

FIG. 24 shows a helical tube of the suture delivery device of FIG. 21.

FIG. 25 shows a sharp distal anchor tip of a barbed suture extending from a distal end of the helical tube of the suture delivery device of FIG. 21.

FIG. 26 shows a port of a leur lock of the suture delivery device of FIG. 21 with a proximal end of the suture held in a groove.

FIG. 27 shows the distal tip of the insertion device of FIGS. 21-26 inserted into a trocar implanted in an incision in a tissue wall with the projections retracted.

FIG. 28 shows the distal tip of the insertion device of FIGS. 21-26 inserted into the incision in the tissue wall with the projections extended.

FIG. 29 shows the projections of the distal tip of the insertion device of FIGS. 21-26 brought into contact with an inner surface of the tissue wall.

FIG. 30 shows the suture delivery device of FIGS. 21-26 slid into position adjacent to an outer surface of the tissue wall.

FIG. 31 shows the helical tube of the suture delivery device of FIGS. 21-26 advanced through the tissue wall.

FIG. 32 shows a syringe connected to the suture delivery device of FIGS. 21-26 for administering liquid medication along the helical tube via holes in the tube.

FIG. 33 shows the syringe removed from the suture delivery device of FIGS. 21-26.

FIG. 34 shows the barbed suture of the suture delivery device of FIGS. 21-26 embedded through the tissue wall with the remainder of the second assembly withdrawn.

FIG. 35 shows the barbed suture of the suture delivery device of FIGS. 21-26 pulled proximally to close the incision.

FIG. 36 shows an exemplary method for closing a port access incision in the tissue wall using the assembly described in FIGS. 1-6.

FIG. 37 shows an alternative embodiment for a suture delivery device comprising a mechanism for ejecting the anchor tip carrying the suture from the helical tube.

FIG. 38 shows the suture delivery device of FIG. 37 coupled to the insertion device of FIG. 22.

FIG. 39 shows the distal tip of the helical tube of FIG. 37 with the anchor tip carrying the suture placed therein.

FIG. 40 shows the suture delivery device of FIG. 37 advanced adjacent to the outer surface of the tissue wall.

FIG. 41 shows the handle of the suture delivery device of FIG. 37 advanced distally along the body so that the helical tube is driven into the tissue wall.

FIGS. 42-43 show cross-sectional views of the suture delivery device of FIG. 37 coupled to the insertion device of FIG. 22 when a positive stop is engaged to prevent the handle from traveling further distally relative to the insertion device.

### DETAILED DESCRIPTION

The exemplary embodiments may be further understood with reference to the following description and the appended drawings, wherein like elements are referred to with the same reference numerals. The exemplary embodiments relate to an assembly and methods for the closure of a tissue opening (e.g., a port access incision). In certain cases, such an incision may be only 1.5 cm in length while the tissue opening penetrates all the way through a full thickness of an abdominal wall (e.g., perhaps 4-7 cm of depth) along which it is desired to bring the opposing surfaces of the tissue together. Getting stiches through such a small (in cross-sectional area) incision and placing them with accuracy over such depths of tissue (i.e., along the length of such a tunnel in tissue) is often very challenging.

The described embodiments facilitate the accurate placement of suturing and ensure that the suture runs circumferentially at multiple depth levels surrounding the defect. Those skilled in the art will understand that these embodiments may be employed to close other tissue openings or defects where the area of the tissue opening is small compared to its depth. For example, embodiments may be useful in preforming deep large-bore core biopsies of the liver, breast, or lung to facilitate hemostasis, eliminate dead space, and/or to decrease air leaks after lung core biopsy.

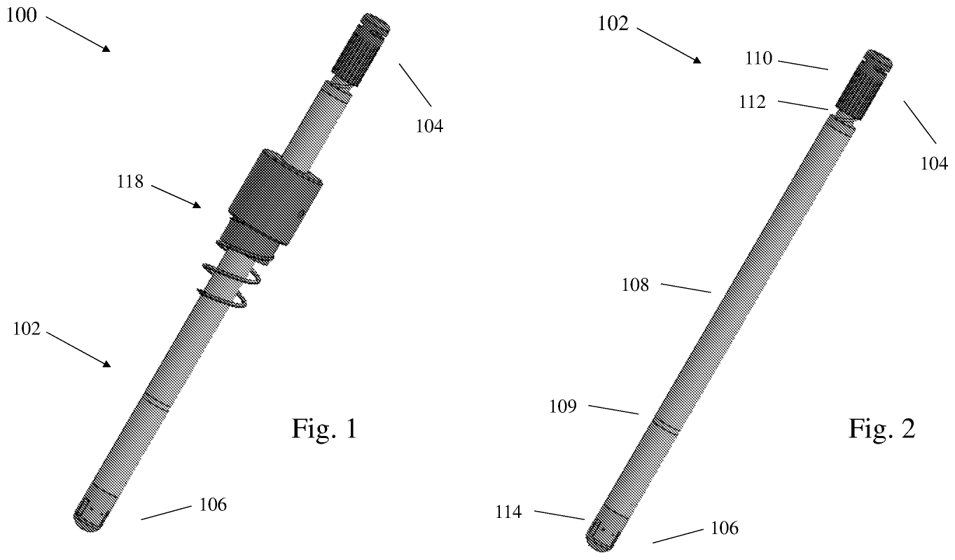

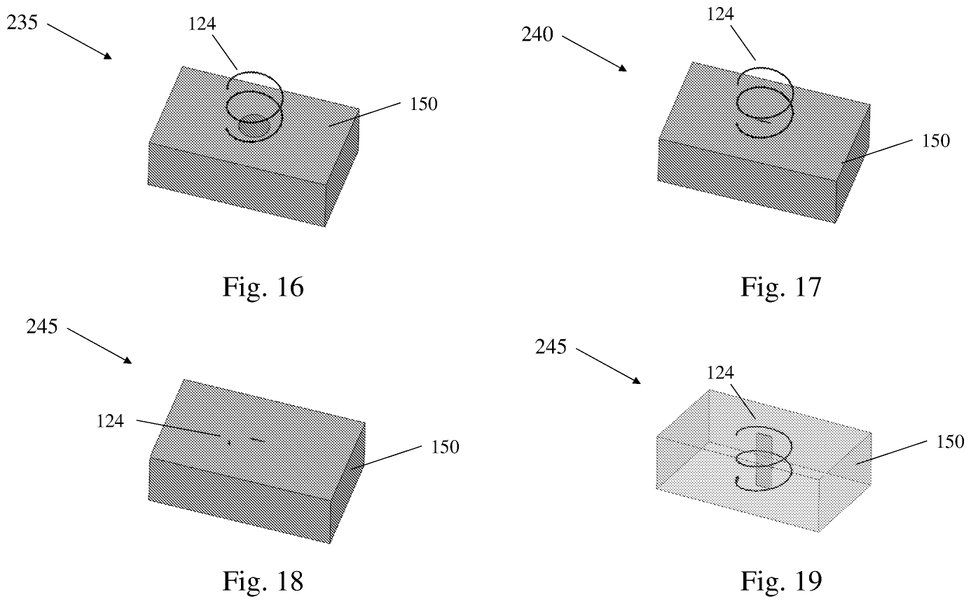

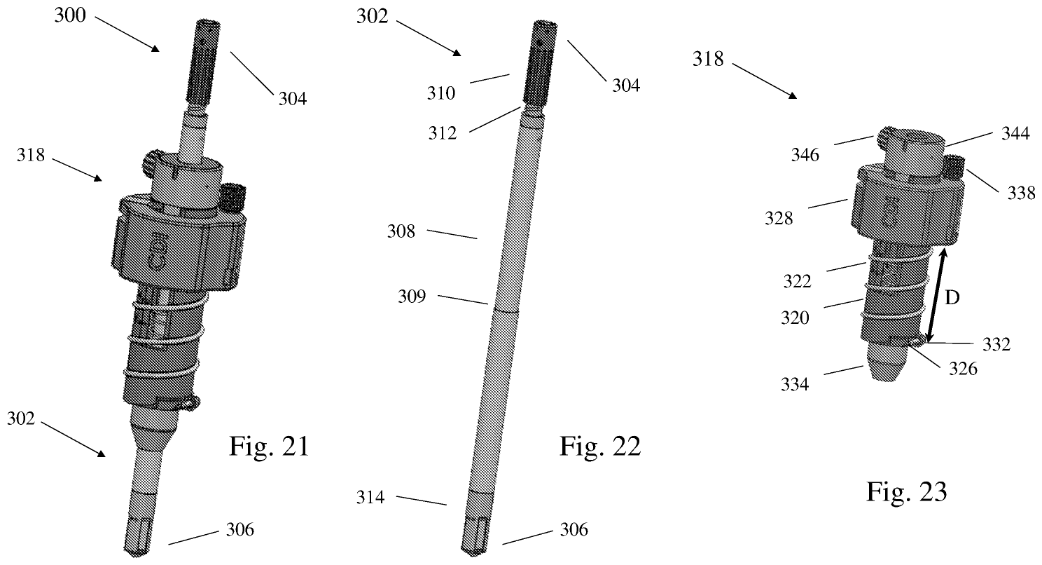

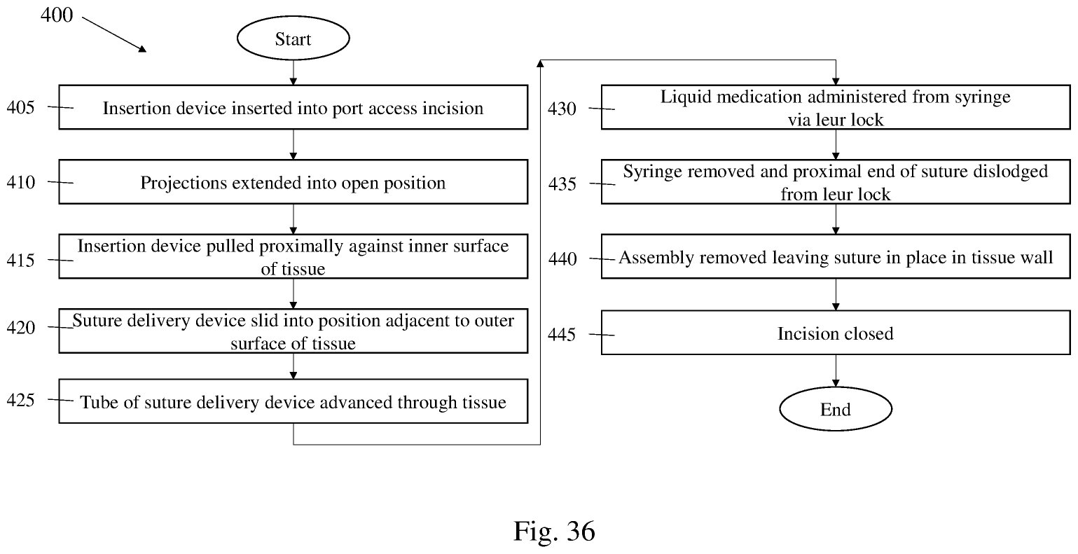

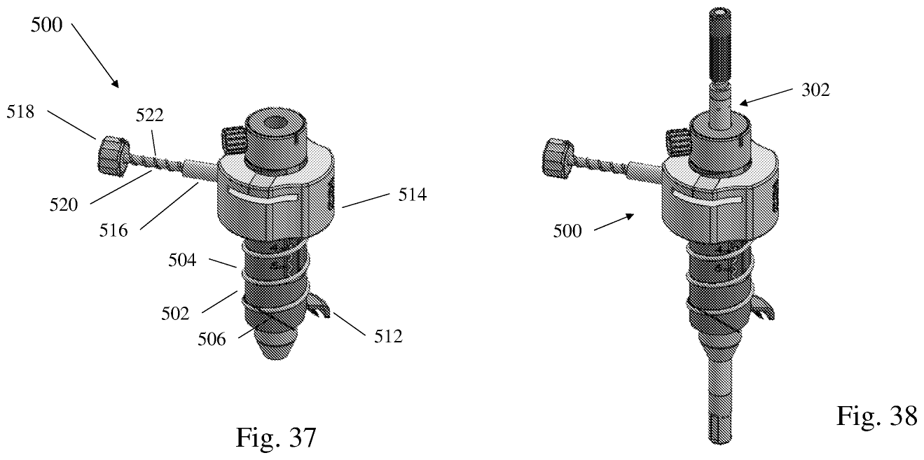

The various exemplary embodiments are described with regard to a first assembly, as shown and described in FIGS. 1-20, and a second assembly, as shown and described in FIGS. 21-36. An alternative suture delivery device similar to that of the second assembly is described in FIGS. 37-43. These assemblies generally include an insertion device and a suture delivery device slidably coupled to the center guide member of the insertion device for advancing a suture around an incision. In some embodiments, the insertion device may be referred to as an obturator or an obturator assembly. Those skilled in the art will ascertain that an obturator may generally refer to a longitudinal member for use during initial placement of a trocar, or port.

A trocar generally comprises a cannula (e.g., tube) and a sharpened tip and may be used in various procedures including, e.g., laparoscopic surgery, to provide an access port into a body cavity. The obturator can be inserted through the cannula of the trocar to support the insertion of the trocar into the body cavity. It should be understood that the assemblies described herein may be designed for use with a trocar. However, this is not required. Further, it should be understood that the term obturator as used herein can refer to any type of insertion device according to these exemplary embodiments and is not limited devices for use with a trocar. For example, the obturators described herein are intended for use in closing incisions rather than for implanting trocars and may be inserted through an incision to be closed via an already-implanted trocar or may be inserted therein without a trocar.

As will be described in greater detail below, the exemplary insertion devices (and/or obturators) can be advanced distally through an incision into a body cavity, whereupon projections can be deployed so that withdrawing the insertion device proximally will engage and lightly grasp the tissue surrounding the incision (e.g., peritoneum). While holding the insertion device in place, the suture delivery device can be advanced over the insertion device so that a helical needle (and/or tube) can be deployed in (e.g., rotated into) in the tissue surrounding the incision. The helical tube includes a suture therein a distal end of which may be anchored in the tissue so that, when the helical needle is withdrawn, the suture remains in place extending through the tissue along the path traversed by the helical needle.

The suture may then be pulled proximally through the tissue to close the incision. In some aspects, the suture delivery device can include a body for slidably coupling to the insertion device and carrying the helical needle. In some aspects, the body of the suture delivery device will include additional features, e.g., a retractable centering aid, a locking mechanism for locking the suture delivery device to the insertion device, a port (e.g., Leur) providing access to the helical tube so that a therapeutic liquid (e.g., a medication) can be applied through the tube, and other features to be described in further detail below. It should be understood that each of the various features described for the first assembly and the second assembly can be used alone or in various combinations with other features and the present disclosure is not limited to the specific arrangements described for the first assembly and the second assembly. In an alternative embodiment for the suture delivery device, to be described below, an anchor ejection mechanism is used to detach the suture from the helical tube.

It should be further understood that in some scenarios the exemplary suture delivery devices can be used with other types of insertion devices. For example, the insertion device can be designed to grasp the interior tissue by some other means prior to advancing the suture delivery device. In another example, for certain parts of the human body, the grasping functionality described for the exemplary insertion devices may not be required prior to advancing the suture delivery device. Similarly, the exemplary insertion devices may be used in other applications and are not limited to use only with the exemplary suture delivery devices.

As shown in FIGS. 1-6, a first exemplary assembly 100 includes an insertion device 102 and a suture delivery device 118. The suture delivery device 118 can be slidably coupled to a longitudinal center guide member 108 of the insertion device 102. The insertion device 102, e.g., obturator, has an atraumatic distal tip 114 configured to be inserted into a body cavity (e.g., the abdomen) through a port access incision (not shown). A plurality of projections 116 are retractable and extendable laterally from the tip 114. The projections 116 may be retracted to lie flush with the surface of the tip 114 when in an insertion/withdrawal configuration (e.g., to minimize a profile of the device to facilitate its insertion or withdrawal from the port access incision).

When the tip 114 has been positioned as desired (e.g., inserted into the body via the port access incision to a desired position) the projections 116 may be extended laterally outward from the tip 114 to gently hold tissue surrounding the port access incision (e.g., tissue at an inner surface 152 of a body cavity). That is, when the tip 114 has been inserted to a position at which the projections 116 are distal of the inner surface 152 of the tissue through which the device 102 has been inserted, the projections 116 are deployed and the physician gently pulls the insertion device 102 proximally to engage the proximal sides of the projections 116 with the inner surface 152 of the tissue. The suture delivery device 118 may then be deployed to provide helical suturing around a circumference of the incision. The user may then close the incision by tensioning the helically threaded suture, as will be explained in detail further below.

As shown in FIG. 2, the insertion device 102 extends from a proximal end 104 including a handle 110 to a distal end 106 including the tip 114. The handle 110 and the tip 114 are coupled so that an actuator at the handle 110 can deploy and/or retract the projections 116 laterally from the tip 114 as desired. When a procedure has been completed, the user may advance the insertion device 102 distally until the projections 116 are out of contact with the inner surface 152 of the tissue and then retract the projections 116 to facilitate withdrawal of the insertion device 102 from the body.

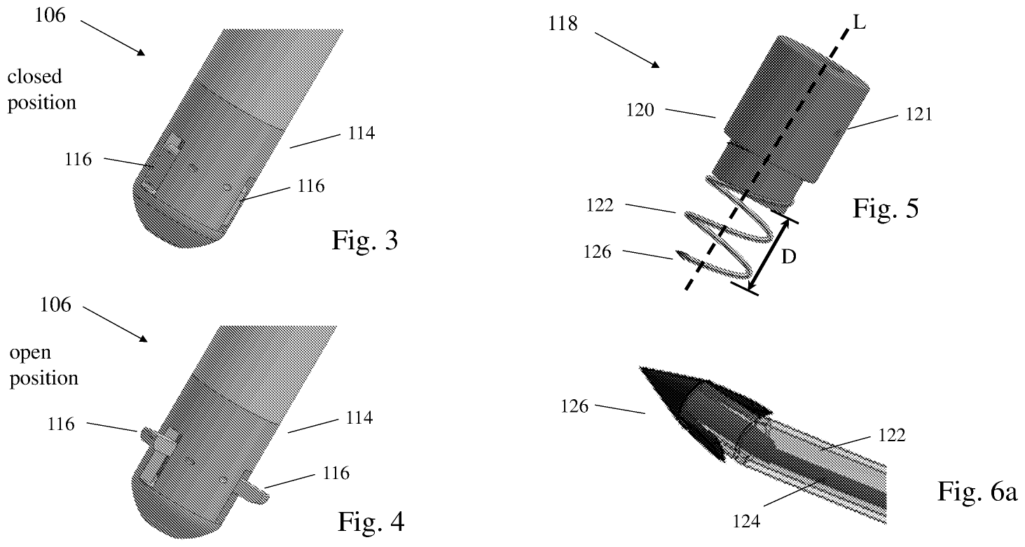

The center guide member 108 includes a channel extending therethrough so that the handle 110 can be coupled to the tip 114 and operate the projections 116 between an open position, in which the projections 116 extend laterally from the tip 114 as shown in FIG. 4 and a closed position in which the projections 116 are retracted flush with the surface of the tip 114 as shown in FIG. 3. In this retracted configuration, the insertion device 102 can be atraumatically inserted into or withdrawn from the body cavity as would be understood by those skilled in the art. In an embodiment, each of the projections 116 may pivot about an axis between the open and the closed configurations. In this example, an inner member 112 is received in the channel of the center guide member 108 and extends between the handle 110 and the tip 114. In this embodiment, rotation of the handle 110 (e.g., by the operating physician) relative to the center guide member 108 moves the handle and the inner member 112 longitudinally within the channel of the center guide member 108 to actuate the projections 116 of the tip 114 to move them between the open and closed configurations.

In one embodiment, a radially inner surface (not shown) of each of the projections 116 is coupled to a distal end of the inner member 112 so that movement of the inner member 112 within the center guide member 108 (i.e., relative to the projections 116) pulls the proximal end of each of the projections 116 radially into the tip 114 deploying the projections 116 laterally outward into the open configuration. Rotation in the opposite direction of the handle 110 and the inner member 112, relative to the center guide member 108, rotates the inner member 112 proximally within the center guide member 108 moving the projections 116 back to the closed (insertion/withdrawal configuration). Although in this embodiment the tip 114 has three projections 116, other embodiments may have any number of projections 116 as long as the projections 116 gently hold tissue surrounding the incision as desired.

In an exemplary embodiment, a spiral projection (not shown) on an outer surface of the inner member 112 mates with a correspondingly shaped groove (not shown) on an inner surface of the center guide member 108 so that rotation of the handle 110 relative to the center guide member 108 moves the inner member 112 longitudinally into or out of the center guide member 108 (depending on the direction of rotation). In an exemplary embodiment, the pitch of the spiral projection and the groove are selected so that clockwise rotation of the handle 110 (e.g., ⅓ turn) moves the inner member 112, e.g., 5 mm along the axis L of the center guide member 108. As the inner member 112 is advanced distally into the center guide member 108, the projections 116 pivot around central pins to the open position. When the handle 110 is rotated counterclockwise relative to the center guide member 108 the center guide member 108 is withdrawn proximally from the center guide member 108, pivoting the projections 116 back into the closed position flush with the surface of the tip 114.

Those skilled in the art will understand that any abutting structure that is deployed laterally outward inside the abdomen may facilitate the precise positioning of a device relative to the abdomen wall (e.g., by allowing the user to pull gently proximally against the peritoneal lining to determine precisely where a deepest plane of the body wall is located). This permits that user to define a geometric plane (e.g., a depth) beyond which the bioabsorbable tissue anchor of the suture delivery device 118, to be described in detail below, is forbidden to travel. That is, the position of the projections 116 defines a maximum depth to which it is desired that the tube 122 and the suture 124 should penetrate the tissue.

The abutting structure that defines that maximum depth may, for example, be a non-compliant balloon, an elastically constrained structure made of Nitinol or polymer, a “Molly” type mechanism that expands radially when foreshortened, or any other number of mechanical solutions.

As shown in FIG. 5, the suture delivery device 118 includes a body 120 that slidably and rotatably couples to the exterior of the center guide member 108 (e.g., an opening through the body 120 is sized and shaped to permit the body 120 to slide over and rotate about the center guide member 108). A helical tube 122, e.g., hypodermic tube (hypo-tube), is fixed to the body 120 (e.g., an interior of the body 120) at a proximal end and extends in a helical shape to a free distal end so that the tube 122 can be rotated about a longitudinal axis L of the body 120 as the body 120 is rotated (in this embodiment the axis of the body 120 is coincident with a longitudinal axis of the center guide member 108 and the inner member 112). Those skilled in the art will understand that the tube 122 is made rigid enough to hold its shape and has column strength sufficient to permit it to be screwed into target tissue (in this example, the abdominal wall). The tube may, for example, be made of stainless steel hypo-tube.

In addition, those skilled in the art will understand that the sharpness of the anchor tip 126 of the suture 124 (described in more detail below) will be designed in consideration of the type of tissue to be penetrated. That is, the type of tissue to be penetrated and within which the suture 124 is to be anchored will dictate the required sharpness of the anchor tip 126 and the structure of its tissue anchoring structures (e.g., barbs).

The tube 122 extends distally from the body 120 for a distance D selected to permit the tube 122 to drive the suture 124 deeply into the tissue surrounding the port access incision without passing out of the inner surface 152 of the tissue wall 150. For example, the tube 122 may comprise at least 2-3 rotations so that the suture may be passed around the circumference of a tissue opening multiple times and the tube 122 extends distally away from a distal end of the body 120 a predetermined distance D (e.g., 4 to 5 cm) along the longitudinal axis of the body 120.

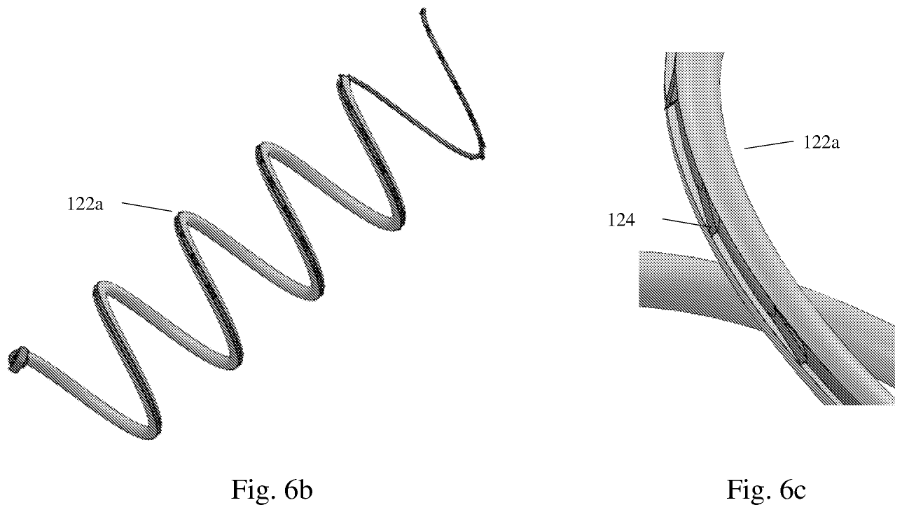

As would be understood by those skilled in the art, the distance D is preferably selected to be slightly less than an expected thickness T of tissue to be penetrated by the tube 122 so that the anchor tip 126 is positioned deeply within the tissue wall 150 without passing through the wall. That is, the distance D is selected so that, when the projections 116 contact the inner surface 152 and the body 120 is advanced until a distal end thereof contacts an outer surface 154 of the tissue wall 150, the distal end of the tube 122 remains within the tissue wall 150. In fact, in a preferred embodiment D is selected so that the anchor tip 126 remains 1-2 mm proximal of the inner surface 152. A helically wound barbed suture 124 is loaded into the tube 122 with a sharp distal anchor tip 126 of the suture 124 extending distally out of the distal end of the tube 122 as shown in FIG. 6a. The suture 124 and the anchor tip 126 can be selected to be bioabsorbable (e.g., Ethicon Stratafix sutures).

In the first exemplary assembly 100 shown in FIGS. 1-6, the insertion device 102 includes a groove 109 (as shown in, e.g., FIG. 2) that is located at a position on the center guide member 108 that allows a mating feature on the body 120 of the suture delivery device 118 to lock into the groove 109 to define a distal-most position of the suture delivery device 118—i.e., to prevent the suture delivery device from moving further distally over the center guide member 108. In other words, the body 120 comprises a hard stop that allows the body 120 to translate distally along the center guide member 108 to a point at which the anchor tip 126 lies in a geometric plane that is a predetermined distance, e.g., 2 mm, above the plane defined by the projections 116 when the projections 116 are extended.

By pulling gently proximally on the insertion device 102 to tighten the tissue around the tip 114, the peritoneal plane (location of the inner surface 152) can be determined. In one example, the mating feature on the body 120 of the suture delivery device 118 comprises a spring-loaded detent located at the small hole 121 at the center of the body 120, as shown in FIG. 5. The detent will lock into the groove 109 at a predetermined distance between the anchor tip 126 and the plane defined by the projections 116 in the open configuration. Thus, by designing the insertion device 102 and the suture delivery device 118 appropriately, the anchor tip 126 is restricted from translating past the peritoneal lining.

In another embodiment, as shown in FIGS. 6b-c, a helical wire 122a comprising a slot is used to carry the suture 124. The slot of this embodiment has a depth sufficient so that the suture 124 is housed below the wire surface to allow smooth penetration of the helical wire 122a when the helical wire 122a is advanced into the tissue.

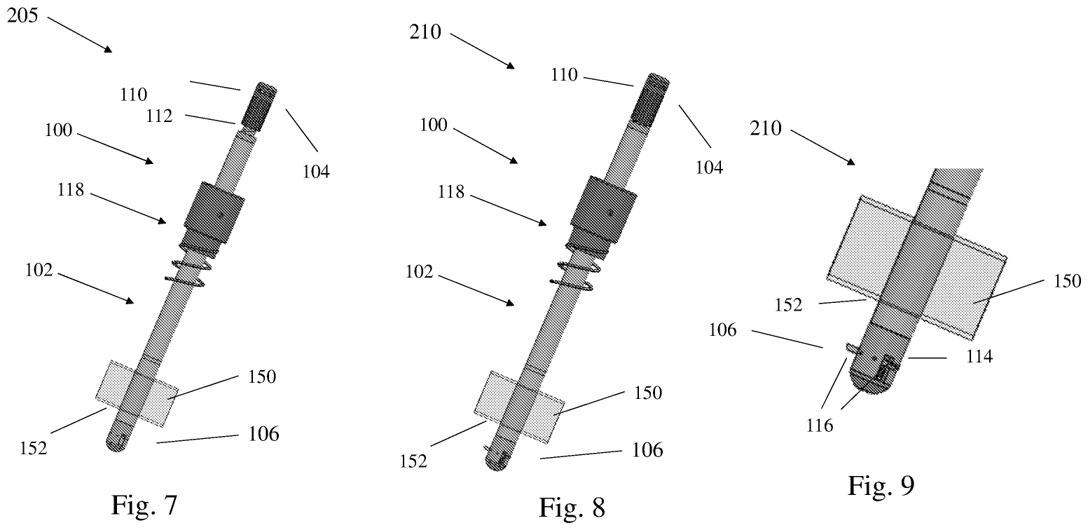

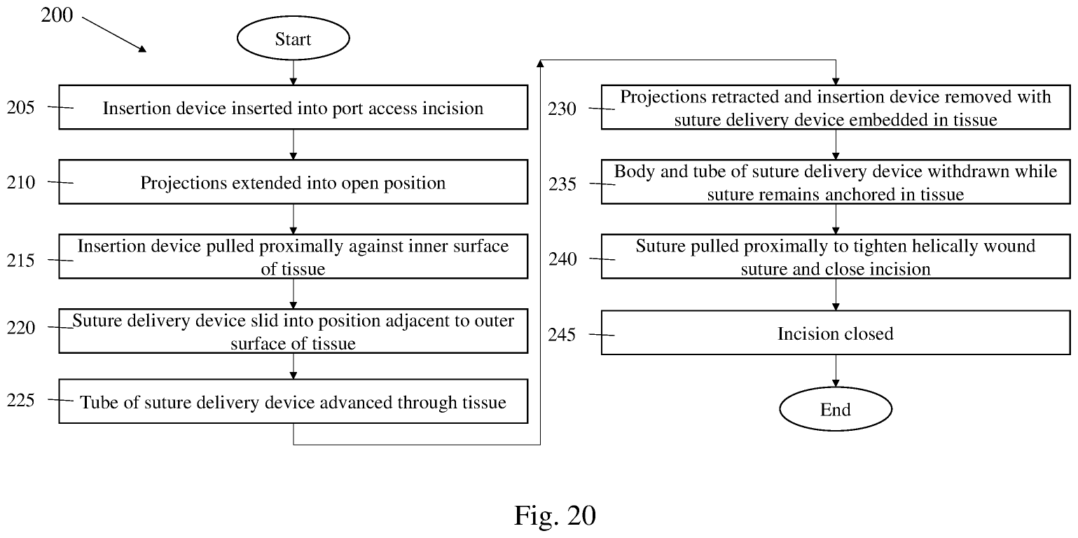

FIGS. 7-19 show an exemplary progression of operations for closing the port access incision in a body tissue wall 150 using the first assembly 100 shown in FIGS. 1-6. FIG. 20 shows an exemplary method 200 for closing the port access incision in the tissue wall 150 using the first assembly 100 and will be described relative to FIGS. 7-19.

In 205, as shown in FIG. 7, the insertion device 102 (i.e., the tip 114 of the insertion device 102) is inserted into the port access incision into the tissue wall 150 with the projections 116 in the closed position. The tip 114 is inserted to a depth sufficient for the projections 116, when extended in the open position, to be positioned distal to the inner surface 152 (e.g., a peritoneal lining) of the tissue wall 150 within the body cavity as would be understood by those skilled in the art.

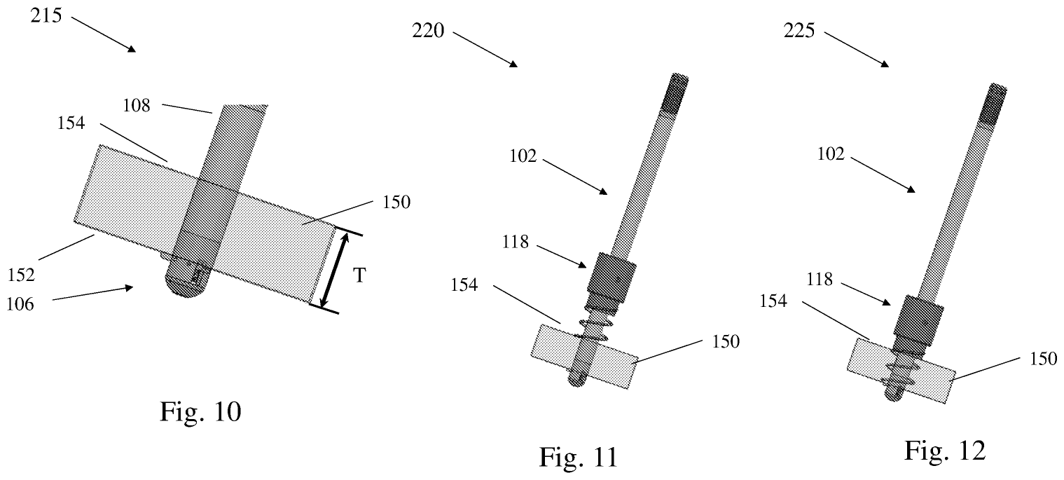

In 210, as shown in FIGS. 8-9, the projections 116 are extended to the open position within the body cavity and the insertion device 102 and is withdrawn proximally (in 215, FIG. 10) to move the projections 116 against the inner surface 152 of the tissue wall 150 to retain the tip 114 in a desired position relative to the tissue. As described above, the physician transitions the projections 116 of this embodiment between the closed and open positions using an actuator on the handle 110. The physician can gently pull on the insertion device 102 proximally after initial contact with the inner surface 152 to ensure that the tissue wall 150 is tightened around the tip 114.

In 220, as shown in FIG. 11, the suture delivery device 118 is slid distally along the center guide member 108 to bring the anchor tip 126 adjacent to an outer surface 154 of the tissue wall 150. In 225, as shown in FIG. 12, the suture delivery device 118 is advanced (e.g., rotated) so that the anchor tip 126 and the tube 122 pierce the outer surface 154 of the tissue wall 150. The tube 122 is rotated about a central axis of the insertion device 102 so that the tube 122 is effectively screwed into the tissue wall 150. The helical shape of the tube 122, in combination with the rotational force (and distal force) applied by the physician, forces the tube 122 and the anchor tip 126 into a large part of the thickness T of the tissue wall 150 (leaving a clearance of, e.g., 1-2 mm between the anchor tip 126 and the inner surface 152) surrounding the port access incision along a helical path.

The tube 122 is advanced distally until the anchor tip 126 reaches the maximum desired depth of insertion into the tissue wall 150 (proximal of the inner surface 152). This desired depth may be reached, for example, when locking features between the center guide member 108 and the suture delivery device 118 are engaged. As described above, this positive stop feature may comprise a spring-loaded detent on the body 120 of the suture delivery device 118 engaging a groove 109 on the center guide member 108. However, other types of stop features can be used as would be understood by those skilled in the art.

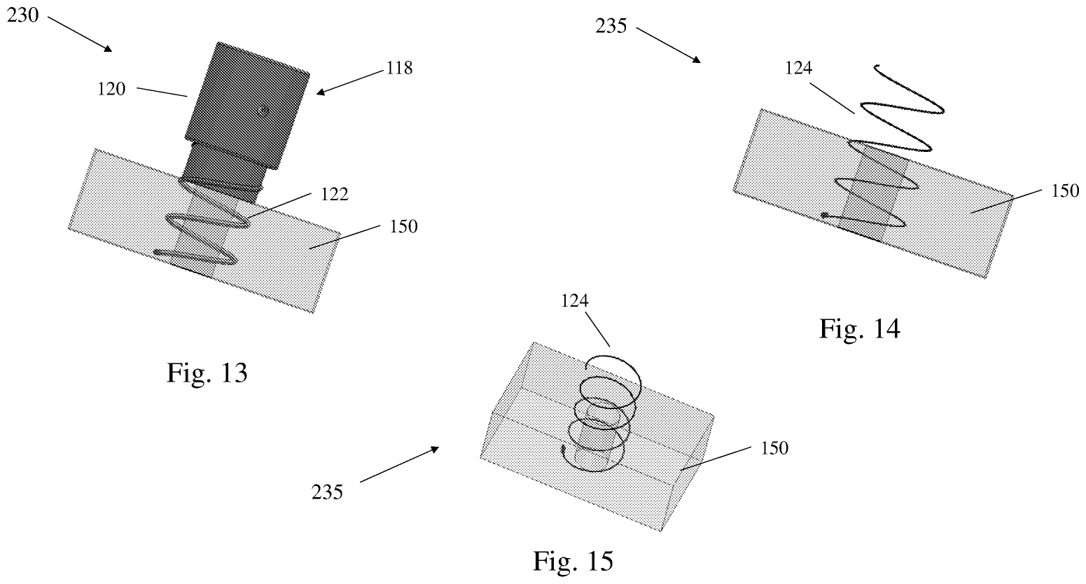

In 230, as shown in FIG. 13, the projections 116 are retracted and the insertion device 102 (in the closed position) is then withdrawn proximally from the suture delivery device 118 (e.g., slide proximally through the opening in the body 120 of the suture delivery device 118) which remains in place with the tube 122 embedded in the tissue wall 150. In 235, as shown in FIGS. 14-16, the body 120 and the tube 122 of the suture delivery device 118 are withdrawn proximally from the tissue (e.g., the body 120 is rotated in the reverse direction to back the tube 122 out of the tissue wall 150 along the previously formed helical path. As the anchor tip 126 of the suture 124 is lodged within the tissue wall 150, as the tube 122 is withdrawn from the tissue wall 150, the suture 124 is pulled out of the tube 122 and remains within the tissue wall 150 (i.e., extending along the helical path formed by the tube 122). In this embodiment, the anchor tip 126 anchors within a distal portion of the tissue wall 150, with a barb or other tissue retention feature holding the suture 124 in place as the tube 122 is withdrawn from the tissue wall 150.

In 240, as shown in FIG. 17, after withdrawal of the tube 122 completely from the suture 124, a proximal portion of the suture 124 extending proximally out of the tissue wall 150 is pulled proximally, tightening the suture 124 within the tissue wall 150 to close the port access incision in step 245, as shown in FIGS. 18-19. The proximal end of the suture 124 may be held in place to maintain the closed state of the incision in any known manner (e.g., by knotting or adhesive, etc.) as would be understood by those skilled in the art. Thus, the anchor tip 126 and the suture 124 hold the incision closed while the incision heals. As would be understood by those skilled in the art, the suture 124 and anchor tip 126 may optionally be formed of material selected to be resorbed into the body so that they need not be removed in a separate procedure.

As shown in FIGS. 21-26, a second exemplary assembly 300 includes an insertion device 302 and a suture delivery device 318. Similar to the first assembly 100, the suture delivery device 318 can be slidably coupled to a longitudinal center guide member 308 of the insertion device 302. The insertion device 302, e.g., obturator, may be similar to the insertion device 102 described with regard to the first assembly 100. The insertion device 302 has an atraumatic distal tip 314 configured to be inserted into a body cavity (e.g., the abdomen) through a port access incision (not shown).

A plurality of projections 316 are retractable and extendable laterally from the tip 314, similar to the tip 114 of the insertion device 102 (as shown, e.g., in FIGS. 3-4). The projections 316 may be retracted to lie flush with the surface of the tip 314 when in an insertion/withdrawal configuration (e.g., to minimize a profile of the device to facilitate its insertion or withdrawal from the port access incision). As shown in FIG. 22, the insertion device 302 extends from a proximal end 304 including a handle 310 to a distal end 306 including the tip 314. The handle 310 and the tip 314 are coupled so that an actuator at the handle 310 can deploy and/or retract the projections 316 configured to extend laterally from the tip 314 as desired.

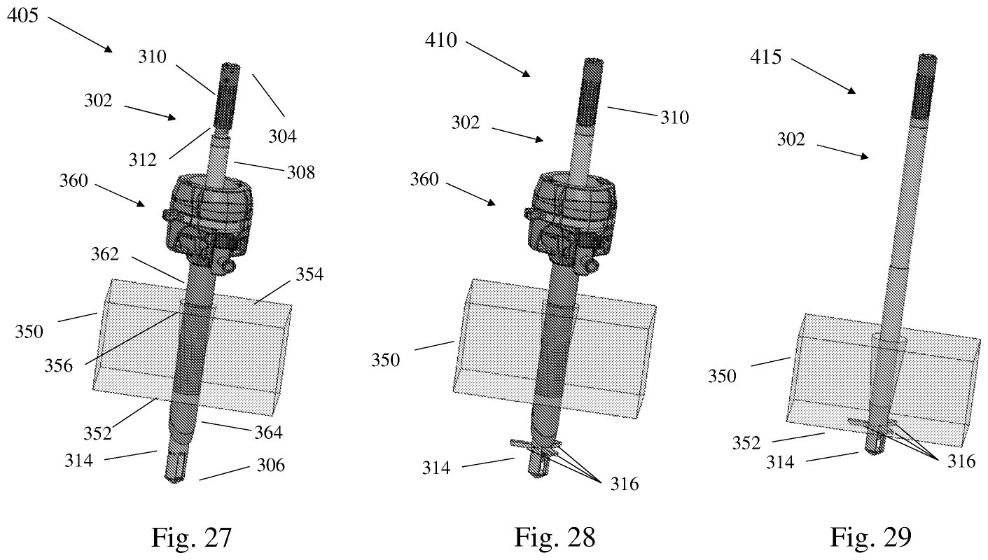

Similar to the insertion device 102 of the first assembly 100 described above, the center guide member 308 includes a channel extending therethrough so that the handle 310 can be coupled to the tip 314 and operate the projections 316 between an open position, wherein the projections 316 extend laterally from the tip 314, as shown, e.g., in FIG. 28, and a closed position in which the projections 316 are retracted flush with the surface of the tip 314, as shown, e.g., in FIG. 27. In some embodiments, an inner member 312 is received in the channel of the center guide member 308 and extends between the handle 310 and the tip 314.

The handle 310 is coupled to the center guide member 308 so that rotation of the handle 310 (e.g., by the operating physician) relative to the center guide member 308, moves the handle and the inner member 312 longitudinally within the channel of the center guide member 308 to actuate the projections 316 of the tip 314 to move them between the open and closed configurations. Although in this embodiment the tip 314 has three projections 316, other embodiments may have any number of projections 316 as long as the projections 316 are configured to gently hold tissue surrounding the incision as desired so that the proper position of the device may be maintained without damaging the tissue. Those skilled in the art will understand that any abutting structure that is deployed laterally outward inside the abdomen may facilitate the precise positioning of a device relative to the abdomen wall (e.g., by allowing the user to pull gently proximally against the peritoneal lining to determine precisely where a deepest plane of body wall is located). This permits that user to define a geometric plane (e.g., a depth) beyond which the bioabsorbable tissue anchor is prevented from penetrating. That is, the position of the projections 316 define a maximum depth to which the tube 322 and the suture 324 will penetrate the tissue.

As shown in FIGS. 21 and 23, the suture delivery device 318 includes a body 320 that slidably couples to the exterior of the center guide member 308, e.g., an opening through the body 320 is sized and shaped to permit the body 320 to slide over the center guide member 308. The body 320 may further be rotatable about the center guide member 308, however, this is not required. In the second assembly 300, the suture delivery device 318 includes multiple additional features, relative to the suture delivery device 118 described above for the first assembly 100. In one aspect, the suture delivery device 318 further includes a handle 328 for delivering a helical tube 322 carrying a suture 324. The handle 328 is slidably and rotatably coupled to the exterior of the body 320, e.g., an opening through the handle 328 is sized and shaped to permit the handle 328 to rotate about and slide over the body 320.

In this example, the tube 322 is fixed to the handle 328 (e.g., an interior of the handle 328) at a proximal end and extends in a helical shape to a free distal end so that the tube 322 is rotated about a longitudinal axis of the handle 328 (and the body 320) as the handle 328 is rotated. The tube 322 of the second assembly 300 may be similar to the tube 122 of the first exemplary assembly 100, e.g., stainless steel hypo-tube sufficiently rigid to hold its shape and having a column strength sufficient to permit it to be driven (screwed) into the tissue wall 350 (e.g., an abdominal wall) as described below in FIGS. 27-36.

The tube 322 extends distally from the body 320 for a distance D selected to permit the tube 322 to drive the suture 324 deeply into the tissue surrounding the port access incision without passing distally out of the inner surface 352 of the tissue wall 350, as shown, e.g., in FIGS. 31-33, similarly to the tube 122 of the first exemplary assembly 100.

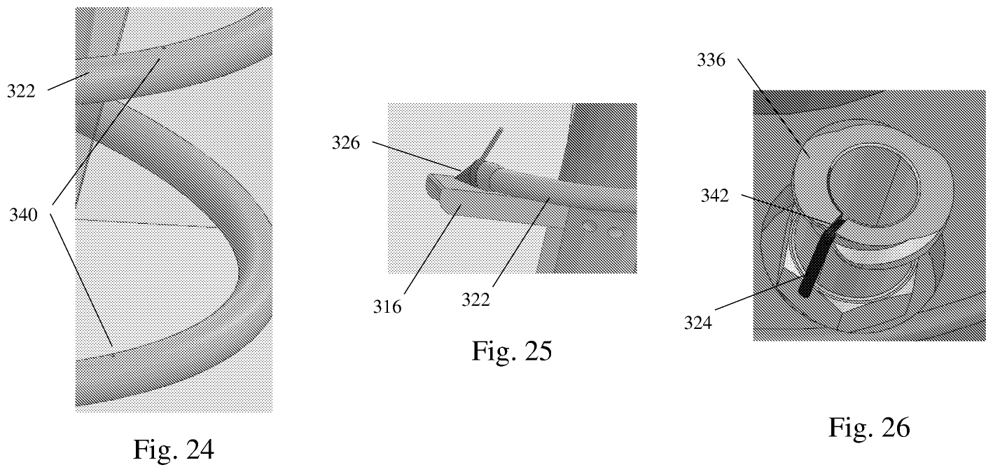

A helically wound barbed suture 324 is loaded into the tube 322, with a sharp distal anchor tip 326 of the suture 324 extending distally out of the distal end of the tube 322, as will be described in greater detail below with regard to FIGS. 24-25. The suture 324 and the anchor tip 326 of this embodiment have been selected to be bioabsorbable (e.g., Ethicon Stratafix sutures). In addition, those skilled in the art will ascertain that the sharpness of the anchor tip 326 of the suture 324 will be designed in consideration of the type of tissue to be penetrated.

In the second assembly 300, the body 320 includes a helical guide 330, e.g., a helical groove on the exterior of the body 320, sized and shaped to slidably receive the tube 322 therein, as shown in FIGS. 31-33. The helical guide 330 provides further support to the tube 322 as the tube 322 is driven into the tissue wall 350, e.g., so that the tube 322 retains its original shape and any deformation is minimized. To further reduce deformation, the body 320 of this embodiment includes a tube support projection 332 at the distal end of the helical guide 330, as shown in FIG. 23.

In further aspects of these exemplary embodiments, the suture delivery device 318 includes additional features for facilitating the suturing of the incision while minimizing trauma to the surrounding tissue and helping to manage the wound post-surgery.

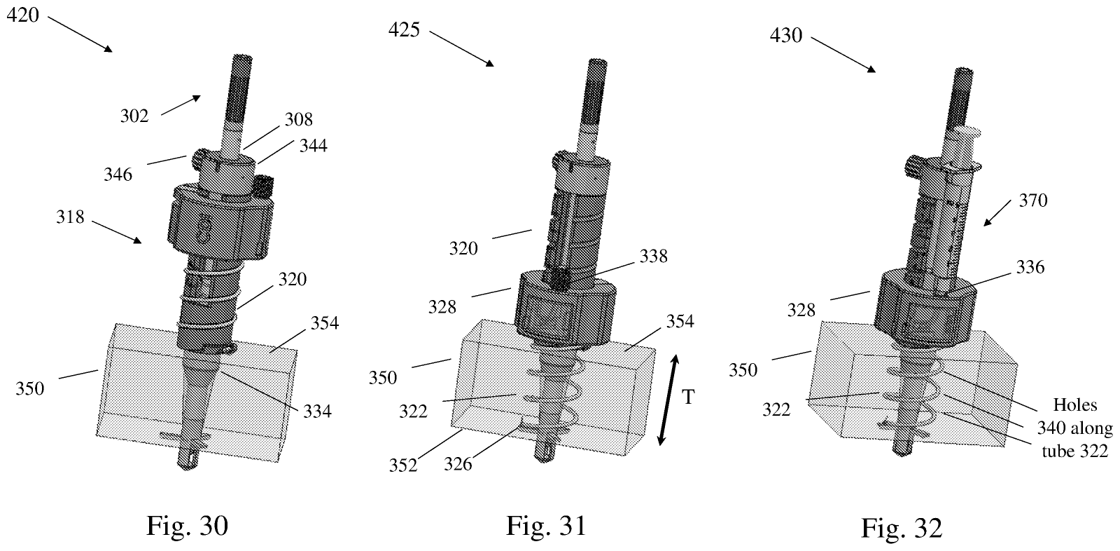

In one aspect, the body 320 includes a retractable centering aid 334 extending from its distal end. The centering aid 334 helps to center the insertion device 302 (e.g., obturator) so that the longitudinal axes of the insertion device 302 and the suture delivery device 318 are directed through the port access incision 356 along a desired path, as will be described in greater detail below with regard to FIG. 30. Thus, the barbed suture, when driven into the tissue wall 350, will encircle the port access incision 356, as will be described in greater detail below with regard to FIG. 31.

In another aspect, the handle 328 includes one or more features for administering medicine to the target anatomy via the tube 322 after the helical tube 322 has been driven into the tissue wall 350 or at any time after the helical tube 322 has been driven into any portion of the tissue. In one embodiment, the handle 328 includes a leur lock comprising a port 336 for attaching a syringe and a cap 338 for protecting the port 336 when not in use, as will be described in greater detail below with regard to FIG. 31-33. The handle 328 of this embodiment includes internal tubing extending between the port 336 and the proximal end of the tube 322, such that liquid medication administered at the port 336 will travel distally down the tube 322. The liquid medication can comprise, e.g., lidocaine or any other therapeutic substance such as a medicine, saline, etc.

As shown in FIG. 24, the tube 322 carrying the suture 324 of one embodiment includes holes 340 along its length at selected locations (e.g., a number of holes 340 equally spaced along the tube 322) so that a therapeutic liquid injected into the tube 322 from the handle 328 will be delivered through the holes 340 to infuse the abdominal tissue wall to help with post surgery wound management.

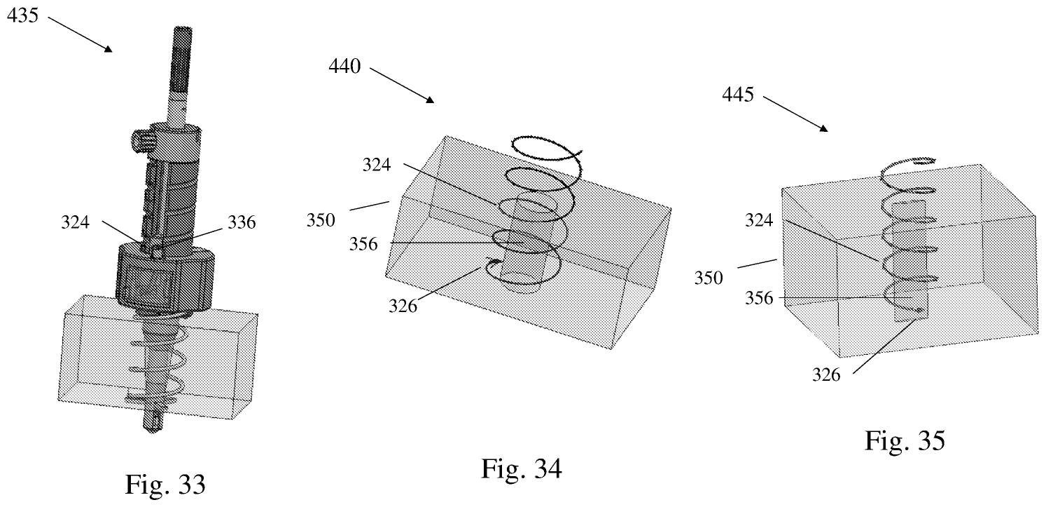

As shown in FIG. 26, a proximal end of the suture 324 (e.g., the free end) can extend through the leur lock so that the proximal end can be dislodged from the suture delivery device 318 after implantation of the suture 324 in the target anatomy. In this example, the port 336 of the leur lock has an internal groove 342 in which the proximal end of the suture 324 can be tightly yet releasably held. When the helical tube 322 has reached its furthest distal position and lodged the anchor tip 326 of the suture 324 in the tissue wall 350 just above the inner surface 352 (e.g., peritoneum) the handle 328 can be withdrawn (rotated in the reverse direction) along the helical path in the tissue wall 350, to be described in greater detail below with regard to FIG. 33.

To allow the suture 324 to remain in the target anatomy when the handle 328 is withdrawn, the proximal end of the suture 324 is dislodged from the internal groove of the port 336 prior to withdrawing the handle 328. Those skilled in the art will ascertain that any suitable release mechanism can be used to detach the suture 324 from the suture delivery device 318, and the arrangement described above using the port 336 of the leur lock is exemplary only.

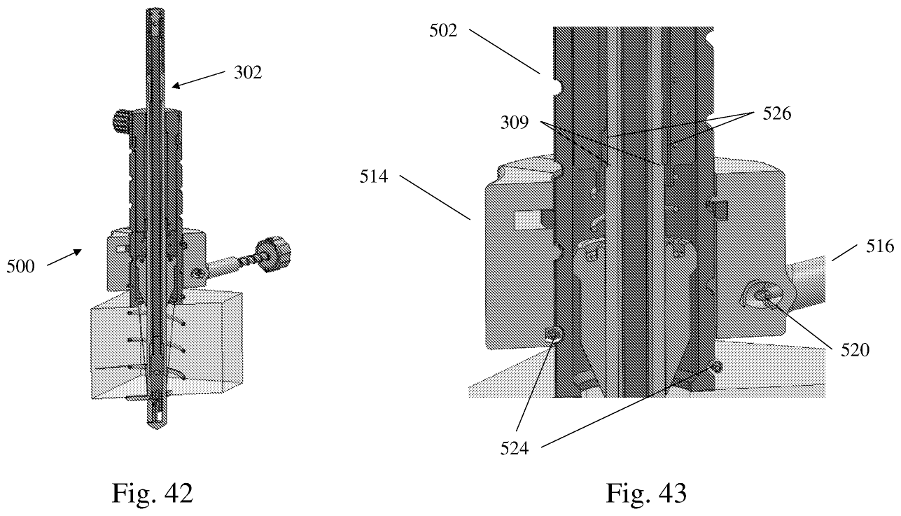

In still another aspect, the second assembly 300 includes one or more features for stopping the tube 322 from advancing past a predetermined furthest distal position relative to the insertion device 302 (obturator), such that the anchor tip 326 does not pierce the inner surface 352 of the tissue wall 350. This feature may be similar to the example described for the first assembly 100 wherein, e.g., the insertion device 302 includes a groove located at a position on the center guide member 308 configured to lockingly receive a mating feature on the handle 328 to prevent any movement of the handle 328 (including the tube 322 and suture 324) further distally along the center guide member 308. In the example shown in FIGS. 21-26, the insertion device 302 includes a step 309 (as shown in, e.g., FIG. 22) that is located at a position on the center guide member 308 that allows a mating feature on the handle 328 of the suture delivery device 318 to contact the step 309 to define a distal-most position of the handle 328. One example of this positive stop mechanism is described in further detail below with respect to FIGS. 42-43.

Those skilled in the art will ascertain that other types of positive stops can be used to restrict the distal motion of the tube 322. As shown in FIG. 25, the tube 322 can be stopped at a position just above the projections 316 of the insertion device 302 so that, when the projections 316 are extended and lightly grasping the inner surface 352 of the tissue wall 350, as will be described in greater detail below with regard to FIG. 31, the anchor tip 326 of the suture 324 does not pierce the inner surface 352.

In still another aspect, the suture delivery device 318 of one embodiment of the second assembly 300 includes one or more features for locking the body 320 to the center guide member 308 of the insertion device 302 at a desired location. In this example, a locking mechanism comprises a clamp portion 344 and a locking knob 346 that can be rotated in a first direction to tighten the clamp portion 344 around a proximal end of the body 320 and rotated in the opposite direction to loosen the clamp portion 344.

When the clamp portion 344 is sufficiently tightened around the body 320 the body 320 will deform such that sliding of the body 320 relative to the center guide member 308 is restricted, e.g., so that the body 320 and the center guide member 308 are effectively fixed to each other. The operating physician can fix the body 320 to the center guide member 308 at a desired location, e.g., when the distal end of the body 320 contacts the outer surface 354 of the tissue wall 350, as shown in FIG. 30. The adjustable locking mechanism allows the anchor tip 326 of the suture 324 to be brought adjacent to the outer surface 354 such that rotating the handle 328 causes the anchor tip 326 to pierce the outer surface 354.

It should be understood that, in this example, the one or more features for stopping the tube 322 from advancing past a predetermined furthest distal position operate regardless of the position at which the body 320 is locked to the center guide member 308. This allows for appropriate placement of the suture regardless of a thickness of the body tissue through which the suture is advanced.

FIGS. 27-35 show an exemplary progression of operations for closing the port access incision 356 in a body tissue wall 350 using the second assembly 300 shown in FIGS. 21-26. FIG. 37 shows an exemplary method 400 for closing the port access incision in the tissue wall 350 using the second assembly 300 and will be described relative to FIGS. 27-35.

In 405, as shown in FIG. 27, the insertion device 302 (the tip 314 of the insertion device 302) is inserted through a trocar 360 placed in the port access incision 356. As described above, the insertion device 302 may be referred to as an obturator. The trocar 360 can comprise any standard trocar having a size suitable for placement in the target anatomy. For example, if the port access incision 356 is in the tissue wall 350, the trocar can be 10 mm or larger. The trocar 360 includes a cannula 362 having a hollow interior and a distal end 364 through which the insertion device 302 (obturator) can be inserted into the port access incision 356 in the tissue wall 350 with the projections 316 in the closed position. The tip 314 is inserted to a depth sufficient for the projections 316, when extended in the open position, to be positioned distal to an inner surface 352 (e.g., a peritoneal lining) of the tissue wall 350 within the body cavity as would be understood by those skilled in the art. As described above, in other embodiments, the insertion device 302 can alternatively be inserted in the target anatomy without the use of a trocar.

In 410, as shown in FIG. 28, the projections 316 are extended to the open position within the body cavity to engage the inner surface 352 of the tissue wall 350 to retain the tip 314 in a desired position. As described above, the physician can transition the projections 316 between the closed and open positions using an actuator on the handle 310, e.g., by rotating the handle 310. In this example, the projections 316 are opened prior to withdrawing the trocar 360 from the port access incision 356. However, this step may be performed after the trocar 360 is withdrawn.

In 415, as shown in FIG. 29, the trocar 360 is removed and the insertion device 302 is pulled proximally by the physician so that the projections 316 are brought into contact with the inner surface 352 of the tissue wall 350. As would be understood by those skilled in the art, the physician may gently pull on the insertion device 302 proximally after initial contact with the inner surface 352 to ensure that the tissue wall 350 is held against and around the tip 314 as desired.

In 420, as shown in FIG. 30, the suture delivery device 318 is placed over the insertion device 302 and slid distally along the center guide member 308 while holding the insertion device 302 to bring a distal end of the body 320 adjacent to an outer surface 354 of the tissue wall 350. In this example, the centering aid 334 extending from the distal end of the body 320 is used to center the insertion device 302 and the suture delivery device 318. When the distal end of the body 320 is in contact with the outer surface 354 of the tissue wall 350, the locking knob 346 is used to tighten the clamp portion 344 to restrict any sliding of the body 320 relative to the center guide member 308.

In 425, as shown in FIG. 31, the handle 328 is advanced (e.g., rotated) relative to the body 320 (e.g., the operating physician can hold the body 320 while driving the handle 328) so that the anchor tip 326 and the tube 322 pierce the outer surface 354 of the tissue wall 350. The tube 322 is rotated about a central axis of the insertion device 302 so that the tube 322 is effectively screwed into the tissue wall 350. The helical shape of the tube 322, in combination with the rotational force (and distal force) applied by the physician, forces the tube 322 and the anchor tip 326 into a large part of the thickness T of the tissue wall 350 (leaving a clearance of, e.g., 1-2 mm between the anchor tip 326 and the inner surface 352) surrounding the port access incision 356 along a helical path. The tube 322 is advanced distally until the anchor tip 326 reaches the maximum desired depth of insertion into the tissue wall 350 (proximal of the inner surface 352) without piercing the inner surface 352, as described above with regard to FIG. 25. This desired depth may be reached when locking features between the center guide member 308 and the suture delivery device 318 are engaged. As described above, this positive stop feature may comprise a spring-loaded detent on the body 320 of the suture delivery device 318 engaging a groove on the center guide member 308. However, other types of stop features can be used.

In 430, as shown in FIG. 32, the cap 338 of the leur lock is removed so that a syringe 370 can be connected to the port 336 of the leur lock. The syringe 370 can be filled with a therapeutic liquid, e.g., lidocaine, that can be administered via the port 336 and tubing internal to the handle 328 into the tube 322. The liquid may be injected through the tube to deliver the medication through the holes 340 along the tube 322 to infuse the tissue wall 350 and facilitate the healing of the tissue wall 350, as described above with regard to FIG. 24.

In 435, as shown in FIG. 33, the syringe 370 is removed after the medication is administered and the proximal end of the suture 324 is dislodged from the internal groove 342 of the port 336 in which the proximal end of the suture 324 is held, as described above with regard to FIG. 26.

In 440, as shown in FIG. 34, the second assembly 300 is removed from the tissue wall 350 leaving the suture 324 in place in the tissue wall 350. The handle 328 is rotated in the reverse direction to withdraw the tube 322 proximally from the tissue wall 350 along the previously formed helical path. As the anchor tip 326 of the suture 324 is lodged within the tissue wall 350, as the tube 322 is withdrawn from the tissue wall 350, the suture 324 is pulled out of the tube 322 (or rather, the tube 322 is pulled away from the suture 324) so that the suture 324 remains within the tissue wall 350 (i.e., extending along the helical path formed by the tube 322). The second assembly 300 including the suture delivery device 318 and the insertion device 302 may then be removed, either by withdrawing the second assembly 300 at once (after retracting the projections 316 of the insertion device 302), or by first using the locking knob 346 to loosen the clamp portion 344 to decouple the suture delivery device 318 from the insertion device 302, withdrawing the suture delivery device 318 from the insertion device 302, and then retracting the projections 316 and withdrawing the insertion device 302 from the port access incision 356.

In 445, as shown in FIG. 35, the proximal portion of the suture 324 extending proximally out of the tissue wall 350 is pulled proximally, tightening the suture 324 within the tissue wall 350 to close the port access incision 356. The proximal end of the suture 324 may then be fixed in place to maintain the closed state of the incision in any known manner (e.g., by knotting or adhesive, etc.) as would be understood by those skilled in the art. Thus, the anchor tip 326 and the suture 324 hold the incision closed while it heals and are eventually absorbed into the body.

In alternative embodiments, the suture delivery device 318 described above for the second exemplary assembly 300 can further include an anchor ejection mechanism that can be actuated after the helical tube is driven to its distal-most position within the tissue wall. At this stage of the procedure, it must be ensured that the anchor tip carrying the suture is embedded in the tissue wall in a manner allowing the helical tube to be withdrawn while the suture remains in place. There is an unexpected challenge in carrying the suture (carried by the helical tube) to its distal-most position within the tissue wall and applying a force sufficient to decouple the suture from the helical tube.

As shown in FIGS. 37-39, a suture delivery device 500 according to an alternative embodiment comprises a mechanism for ejecting the anchor tip 508 carrying the suture 506 from the helical tube 504. The suture delivery device 500 can be used in an assembly with the insertion device 302 described above for the second exemplary assembly 300. Certain features of the suture delivery device 500 according to the present example are common to the suture delivery device 318 of the second assembly 300 and will not be described in further detail in the present description. The additional features of the present suture delivery device 500 include an actuating knob 518 that can cause a distal force to be applied to the anchor tip 508 so that the anchor tip 508 is dislodged from the sharp distal tip 510 of the helical tube 504. This distal force is applied via a series of components of the suture delivery device 500 to be described in detail below.

The suture delivery device 500 includes a body 502 that slidably couples to the exterior of the center guide member 308 of the insertion device 302 (as described above in, e.g., FIG. 22) and further includes a handle 514 slidably and rotatably coupled to the exterior of the body 502 for delivering a helical tube 504 carrying a suture 506, similar to the suture delivery device 318 described above.

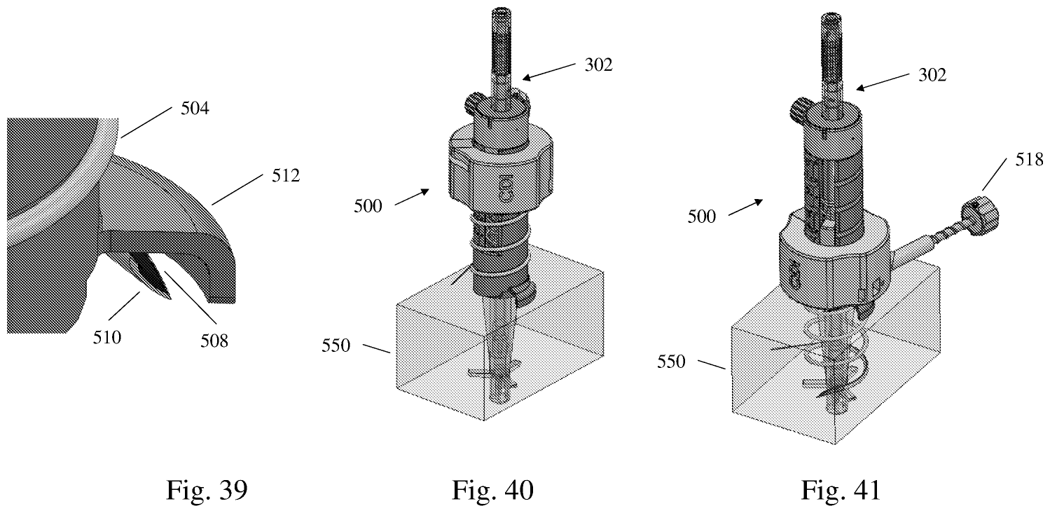

In this example, the tube 504 is fixed to the handle 514 (e.g., an interior of the handle 514) at a proximal end and extends in a helical shape to a free distal end that comprises a sharp distal tip 510 (bevel tip), as shown in FIG. 39. The tube 504 of the present example may be similar to the tube 322 of the second exemplary assembly 300, e.g., stainless steel hypo-tube sufficiently rigid to hold its shape and having a column strength sufficient to permit it to be driven (screwed) into the tissue wall 550 (e.g., an abdominal wall) as described below in FIGS. 40-41. The tube 504 extends distally from the body 502 for a distance selected to permit the sharp distal tip 510 of the tube 504 to drive deeply into the tissue surrounding the port access incision without passing distally out of the inner surface of the tissue wall 550.

In this example, the anchor tip 508 of the suture 506 is loaded into the sharp distal tip 510 of the tube 504, as shown in FIG. 39. For example, the distal tip 510 of the tube 504 can be beveled so that an inner surface of the distal tip 510 can retain the anchor tip 508 of the suture 506. The anchor tip 508 should fit into the distal tip 510 of the tube 504 tightly enough so that the anchor tip 508 is retained as the distal tip 510 is being driven into the tissue wall 550, yet loosely enough so that a force applied in the distal direction to the proximal end of the anchor tip 508 can force the anchor tip 508 out of the distal tip 510 of the tube 504. The body 502 has a projection 512, as shown in FIG. 39, that can help to guide the tube 504 as it is driven into the tissue wall 550. In this example, the suture 506 remains outside the body 502 of the suture delivery device 500, as shown in FIG. 37. Thus, as the distal tip 510 of the tube 504 is driven into the tissue wall 550, the anchor tip 508 of the suture 506 is pulled along with the tube 504 along the helical path through the tissue wall 550.

The anchor ejection mechanism of the present example includes the actuating knob 518, a shaft 520 of the actuating knob 518, and an external wire 522 wrapped around the shaft 520. The shaft 520 can have a threaded distal end (not shown) designed to mate with a corresponding thread in a channel 516 extending off the handle 514, e.g., extending orthogonally from the handle 514 to provide access to the interior of the handle 514. Within the handle 514, the external wire 522 wrapping (e.g., helically) around the shaft 520 can couple to an internal wire 524 (as shown, e.g., in FIG. 43 below) that is sized and shaped to be received within the helical tube 504. The internal wire 524 can slide within the helical tube 504, e.g., distally, when a force is applied on its proximal end. The external wire 522 may be, e.g., aluminum, while the internal wire 524 may be, e.g., Nitinol.

Thus, according to the present example, the suture delivery device 500 comprising the anchor ejection mechanism operates as follows. As shown in FIG. 40, the insertion device 302 can be inserted and the suture delivery device 500 can be brought adjacent to the tissue, whereupon the suture delivery device 500 can be locked to the insertion device 302, as described in previous examples. It is noted that the actuating knob 518 is not shown in FIG. 40 (because it is extending rearward and is blocked by the body 502). As shown in FIG. 41, the handle 514 can be rotated about the body 502 so that the handle 514 moves distally and the sharp distal tip 510 of the helical tube 504 is driven distally into the tissue wall 550. The anchor tip 508 of the suture 506 is loaded into the distal tip 510 of the tube 504, as described above, so the suture 506 follows the helical path made by the tube 504. The handle 514 is rotated until a positive stop feature is engaged between the insertion device 302 and the suture delivery device 500, one example of which to be described in greater detail below with regard to FIGS. 42-43.

When the handle 514 is at its distal-most position, the user can actuate the anchor ejection mechanism of the suture delivery device 500 to detach the anchor tip 508 from the distal tip 510 of the tube 504. The actuating knob 518 is rotated so that the knob 518 travels inwards toward the center of the body 502. This motion of the knob 518 causes the external wire 522 to rotate and/or travel distally therewith, which applies a distal force to the internal wire 524. The internal wire 524 is forced distally through the helical tube 504 so that a distal force is applied to anchor tip 508 of the suture 506. When sufficient force is applied, the anchor tip 508 will eject from the distal tip 510 of the tube 504, thus embedding the anchor tip 508 in the tissue wall 550.

FIGS. 42-43 show cross-sectional views of the suture delivery device 500 in which the handle 514 has reached its distal-most position relative to the insertion device 302. As previously described, the insertion device 302 comprises a step 309 at which the outer diameter of the center guide member 308 increases. Internal to the body 502 of the suture delivery device 500 is a channel through which a cylinder 526 can travel longitudinally. The cylinder 526 is coupled to the handle 514 through the body 502 such that the cylinder 526 travels through the channel as the handle 514 is rotated distally. When the handle 514 reaches a certain position relative to the insertion device 302 the cylinder 526 engages with the step 309 so that the handle 514 cannot travel further distally. Thus, the handle 514 is prevented from traveling so far distally that the distal tip 510 of the helical tube 504 passes the projections 316 in the extended state, e.g., so that the inner surface of the tissue wall 550 is not pierced.

Thus, regardless of the thickness of the tissue wall 550, the distal tip 510 of the tube 504 is prevented from traveling past the projections 316 holding the interior surface of the tissue wall 550. Thus, when the anchor tip 508 is ejected from the distal tip 510, the anchor tip 508 is embedded in the tissue wall 550.

It will be appreciated by those skilled in the art that various modifications and alterations of the disclosed embodiments may be made without departing from the broad scope of the invention. Some of these have been discussed above and others will be apparent to those skilled in the art. For example, though embodiments herein are described as suited for closing port access incisions through the abdominal wall or thoracic wall, as are typically used for port placement in thoracoscopic, laparoscopic, and robotic procedures, these embodiments may also be used to facilitate closure of any tissue opening/defect in which the aspect ratio (wherein a depth of the opening is large compared to its the radius) would make closure by any other means challenging and labor intensive.

What is claimed is:

1. An assembly for closing a tissue opening, comprising:
an insertion device including a longitudinal guide member extending from a proximal end to a distal tip, the distal tip including an abutting structure movable between an insertion position in which the abutting structure is flush with the distal tip and an open position in which the abutting structure is extended laterally from the distal tip, the abutting structure being sized and shaped to contact tissue surrounding the tissue opening into which the insertion device has been inserted to maintain a desired position of the insertion device within the tissue opening; and
a suture delivery device including a body and a tube, the tube including a suture channel extending therethrough, the tube being translatable and rotatable relative to the longitudinal guide member, the tube extending along a curved path configured so that, as the tube is rotated about the longitudinal guide member, the tube penetrates tissue surrounding the tissue opening into which the longitudinal guide member has been inserted, a distal extension of the tube being configured so that, when the tube has been advanced distally to a distal-most position on the longitudinal guide member, a distal end of the tube is proximal of the abutting structure.

2. The assembly of claim 1, further comprising:
a suture slidably received within the tube, the suture including a tissue anchoring distal tip extending distally out of the distal end of the tube, so that, as the tube is introduced into tissue, the tissue anchoring distal tip is driven into the tissue and retained in the tissue to anchor the suture in the tissue as the tube is withdrawn therefrom so that tightening the suture closes the tissue opening.

3. The assembly of claim 1, wherein the insertion device further comprises an actuator coupled to the abutting structure so that movement of the actuator in a first direction relative to the longitudinal guide member extends the abutting structure laterally into the open position and movement of the actuator in a second direction relative to the longitudinal guide member withdraws the abutting structure laterally into a closed position.

4. The assembly of claim 2, wherein the tube is configured so that, as the tube is advanced distally to the distal-most position relative to the longitudinal guide member, the tube is advanced through the tissue around the tissue opening around a full circumference of the tissue opening so that pulling the suture proximally while the suture is anchored in the tissue, produces a multi-layer helical closure of the tissue opening.

5. The assembly of claim 1, wherein the body includes a channel extending therethrough sized and shaped to slidably and rotatably receive the longitudinal guide member therein.

6. The assembly of claim 5, wherein the tube extends distally from the suture delivery device for a distance selected to ensure that, when the tube is inserted into tissue until an aspect of the suture delivery device contacts an outer surface of the tissue, the distal end of the tube will have penetrated the tissue to a desired depth that is proximal of the abutting structure.

7. The assembly of claim 2, wherein the suture includes a tissue anchoring barb at a distal end thereof.

8. The assembly of claim 3, wherein the actuator is coupled to an inner member slidably received within the longitudinal guide member, a distal end of the inner member being coupled to the abutting structure so that movement of the inner member relative to the longitudinal guide member moves the abutting structure between the open and closed positions.

9. The assembly of claim 8, wherein the abutting structure includes a plurality of projections rotatably coupled to the distal tip for rotating between the open and closed positions.

10. The assembly of claim 9, wherein the tube is helical and extends at least twice around a circumference of the body.

11. The assembly of claim 1, wherein the body of the suture delivery device is slidably coupled to the insertion device, the suture delivery device further comprising a handle slidably and rotatably coupled to the body, the tube being fixed to the handle.

12. The assembly of claim 11, wherein the body further comprises a helical guide recess for receiving the tube prior to and during implantation of the tube into the tissue.

13. The assembly of claim 1, wherein the suture delivery device further comprises a port into which liquid medication can be administered, the port connecting to the tube so that the liquid medication travels through the tube and is released into the tissue via holes in the tube.

14. The assembly of claim 1, wherein the suture delivery device further comprises a clamp for locking the suture delivery device at a location along the insertion device via a locking knob.

15. The assembly of claim 1, wherein the suture delivery device comprises a mechanism for detaching a tissue anchoring distal tip carrying a suture from the tube in which the tissue anchoring distal tip is carried.

16. A method for closing a tissue opening, comprising:
inserting into the tissue opening an insertion device including a longitudinal guide member extending from a proximal end to a tip at a distal end thereof with an abutting structure of the tip in an insertion position in which the abutting structure is flush with the distal tip until the tip has passed fully through a thickness of the tissue surrounding the tissue opening;
after the tip has passed fully through the thickness of the tissue surrounding the tissue opening, moving the abutting structure to an open position in which the abutting structure is extended laterally away from the tip;
withdrawing the longitudinal guide member proximally until the abutting structure contacts an inner surface of the tissue surrounding the tissue opening;
rotating and moving distally along the longitudinal guide member a tube extending distally from a suture delivery device along a curved path so that, as the tube is rotated relative to the longitudinal guide member, the tube penetrates tissue surrounding the tissue opening, a distal extension of the tube being configured so that, when the tube has been advanced distally to a distal-most position on the longitudinal guide member, a distal end of the tube is proximal of the abutting structure, the tube including a suture slidably received therein and including a tissue anchor at a distal end thereof; and
withdrawing the tube from the tissue while the tissue anchor remains lodged in the tissue so that, as the tube is withdrawn from the tissue, the suture slides out of the tube to remain within the tissue around the tissue opening.

17. The method of claim 16, further comprising:
pulling the suture proximally to close the tissue opening.

18. The method of claim 16, further comprising:
after the tip has passed fully through the thickness of the tissue surrounding the tissue opening, moving an actuator coupled to the abutting structure in a first direction relative to the longitudinal guide member to extend the abutting structure laterally into the open position.

19. The method of claim 18, further comprising:
after rotating and moving a body of the suture delivery device distally along the longitudinal guide member to advance the tube into the tissue, moving the actuator in a second direction relative to the longitudinal guide member to withdraw the abutting structure laterally into a closed position.

20. The method of claim 16, wherein as the tube is advanced distally to the distal-most position relative to the longitudinal guide member, the tube is advanced through the tissue around the tissue opening around a full circumference of the tissue opening.
